(19) United States
(12) Patent Application Publication
(10) Pub. No.: US 2020/0105416 A1
(43) Pub. Date: Apr. 2, 2020

(54) HEALTH MANAGEMENT APPARATUS, HEALTH MANAGEMENT METHOD, AND STORAGE MEDIUM

(71) Applicants: OMRON HEALTHCARE CO., LTD., Muko-shi (JP); OMRON Corporation, Kyoto-shi (JP)

(72) Inventors: Fumihiko NAKAMURA, Kyoto (JP); Daisuke NOZAKI, Kyoto (JP); Yasushi MATSUOKA, Kyoto (JP)

(21) Appl. No.: 16/699,912

(22) Filed: Dec. 2, 2019

Related U.S. Application Data
(63) Continuation of application No. PCT/JP2018/022225, filed on Jun. 11, 2018.

(30) Foreign Application Priority Data
Jun. 19, 2017 (JP) 2017-119992

(51) Int. Cl. G16H 50/30; A61B 5/021; A61B 5/00; G16H 10/60; G06Q 30/02; G16H 40/67; G16H 20/00

(52) U.S. Cl. CPC G16H 50/30; A61B 5/021; A61B 5/486; G16H 20/00; G06Q 30/0239; G16H 40/67; G16H 10/60

(57) ABSTRACT

A health management apparatus includes an acquisition unit configured to acquire biological information of a measurement subject, a determination unit configured to determine whether or not the acquired biological information attains a goal value set with respect to the biological information within a predetermined period of time, and a calculation unit configured to calculate an incentive value of the measurement subject based on a determination result obtained by the determination unit.

### CROSS-REFERENCE TO RELATED APPLICATIONS

This application is a Continuation Application of PCT Application No. PCT/JP2018/022225, filed Jun. 11, 2018 and based upon and claiming the benefit of priority from Japanese Patent Application No. 2017-119992, filed Jun. 19, 2017, the entire contents of all of which are incorporated herein by reference.

### FIELD

The present invention relates generally to a health management apparatus, a health management method, and a storage medium.

### BACKGROUND

Various approaches to the promotion of health maintenance for a person who becomes a subject of measurement through the use of a health care device have been proposed.

In Jpn. Pat. Appln. KOKAI Publication No. 2003-141260, a health point bank system is proposed. In the health point bank system in Jpn. Pat. Appln. KOKAI Publication No. 2003-141260, if a measurement subject (who is a subject of measurement) uses a health care device, such as a pedometer, the health care device converts biological information obtained as a measurement result into reward points in a predetermined manner, and transmits the reward points to a server. The server then stores the transmitted reward points for each user. The measurement subject receives products and services in accordance with the reward points stored in the server. According to this health point; bank system, it is possible to help the measurement subject to continue behaviors effective for maintaining their health with the use of a health care device.

### SUMMARY

A health management apparatus according to one aspect of the present invention includes an acquisition unit configured to acquire biological information of a measurement subject, a determination unit configured to determine whether or not the acquired biological information attains a goal value set with respect to the biological information within a predetermined period of time, and a calculation unit configured to calculate an incentive value of the measurement subject based on a determination result obtained by the determination unit,

According to the above structure, a measurement subject is able to not only provide measured biological information but also obtain more incentives through attaining a goal value. As a consequence, a measurement subject comes to aim at attaining a goal value to obtain more incentives, and eventually start health management on their own. It is thus possible to accustom a measurement subject to measuring their biological information, and to improve their health situation.

According to the health management apparatus according to the above-described aspect, the determination unit further determines whether or not the acquired biological information is valid, and the calculation unit calculates the incentive value of the measurement subject further based on a number of biological information items that have been determined to be valid by the determination unit. According to the structure, a measurement subject is able to not only obtain incentives in accordance with whether or not a goal value is attained within a predetermined period of time, but also obtain more incentives through acquisition of the increased number of biological information items during the process of attaining a goal value. It is thus possible to motivate a measurement subject to continuously measure their biological information.

According to the health management apparatus according to the above-described aspect, the acquisition unit further acquires health information indicating a health situation of the measurement subject, the determination unit further determines whether or not the acquired biological information satisfies a predetermined condition which is based on the acquired health information, and the calculation unit calculates the incentive value of the measurement subject further based on a number of biological information items determined to satisfy the predetermined condition by the determination unit. According to the structure, a measurement subject can obtain more incentives through providing biological information items which are of particular importance among all the acquired biological information items. For example, if a measurement subject has a symptom of workplace hypertension, a blood pressure value acquired during work tends to be high, and such a value may be particularly important among biological information acquired from the measurement subject. The calculation unit calculates more incentive values for blood pressure values acquired during work for a measurement subject who has a symptom of workplace hypertension. As a consequence, a measurement subject comes to measure their blood pressure during work to obtain more incentives, and eventually start health management on their own. It is thus possible to accustom a measurement subject to measuring their particularly important biological information, and to effectively promote improvement in their health situation.

According to the health management apparatus according to the above-described aspect, the goal value includes at least one intermediate goal value set correspondingly to a point: of time in the predetermined period of time, and

the determination unit further determines whether or not the acquired biological information attains the intermediate goal value at the point of time. According to the structure, a measurement subject is able to not only obtain incentives in accordance with whether or not a goal value is attained within a predetermined period of time, but also obtain more incentives through attaining an intermediate goal value during the process of attaining a goal value. It is thus possible to accustom a measurement subject to measuring their biological information and to promote improvement in their health situation.

According to the health management apparatus according to the above-described aspect, the biological information includes at least one of a blood pressure value and a body weight. According to the structure, a blood pressure value and a body weight of a measurement subject can be targeted for health management. It is thus possible to accustom a measurement subject to measuring their blood pressure value and body weight. It is also possible to motivate a measurement subject to bring their blood pressure value and body weight closer to a goal value.

According to the health management apparatus according to the above-described aspect, further includes an issuance unit configured to issue at least one of reward points and a coupon to the measurement subject based on the calculated incentive value. According to the structure, reward points or a coupon(s) can be issued to a measurement subject by the health management apparatus in accordance with an incentive value. For this reason, a measurement subject can receive specific benefit as a reward for attaining a goal value. It is thus possible to reinforce the motivation to attain a goal value.

According to the present invention, technologies can help build a routine of biological information measurement and promote improvement in a health situation.

### BRIEF DESCRIPTION OF THE DRAWINGS

FIG. 1 is a block diagram showing a configuration of a health management system including a health management apparatus according to an applied example.

FIG. 2 is a block diagram showing a hardware configuration of a biological information measurement apparatus according to an embodiment.

FIG. 3 is a block diagram showing a hardware configuration of a measurement subject's device according to the embodiment.

FIG. 4 is a block diagram showing a hardware configuration of a health management apparatus according to the embodiment.

FIG. 5 is a diagram showing goal management information according to the embodiment.

FIG. 6 is a block diagram showing a hardware configuration of a provider's server according to the embodiment.

FIG. 7 is a block diagram showing a function configuration of the health management apparatus according to the embodiment.

FIG. 8 is a flow chart showing a procedure of a health management process in the health management apparatus according to the embodiment.

FIG. 9 is a flow chart showing a procedure of a health management process in a health management apparatus according to a first modification of the embodiment,

FIG. 10 is a diagram showing goal management information according to a second modification of the embodiment.

FIG. 11 is a flow chart showing a procedure of a health management process in a health management apparatus according to a second modification of the embodiment.

FIG. 12 is a block diagram showing a hardware configuration of a biological information measurement apparatus according to a third modification of the embodiment.

FIG. 13 is a block diagram showing a function configuration of a health management apparatus according to the third modification of the embodiment.

FIG. 14 is a table showing a relationship between a health situation of a measurement subject and a time of day at which blood pressure becomes high according to the third modification of the embodiment.

FIG. 15 is a flow chart showing a procedure of a health management process in a health management apparatus according to the third modification of the embodiment.

FIG. 16 is a diagram showing biological information according to a fourth modification of the embodiment.

### DETAILED DESCRIPTION

As in the health care point bank system described in Jpn. Pat. Appln. KOKAI Publication No. 2003-141260, motivation to use a health care device can be promoted through conversion of biological information measured by a health care device into reward points, and health management of a measurement subject can thus be improved.

Use of a health care device to continue measuring biological information is not easy for a measurement subject who is not habituated to such a device.

In one aspect, the present embodiments are proposed in consideration of such a circumstance and so as to provide technologies which help accustom a measurement subject to measuring their biological information, and to promote improvement in, their health situation.

Hereinafter, embodiments will be described with reference to the drawings. In the descriptions below, constituent elements having the same functions and configurations will be denoted by the same reference symbols. Where there is a need to distinguish constituent elements having the same reference numerals, additional symbols are appended to the reference numerals. If no distinction is necessary, the constituent elements are referred to by the same reference numerals only, and no additional symbols are appended.

### 1. Applied Example

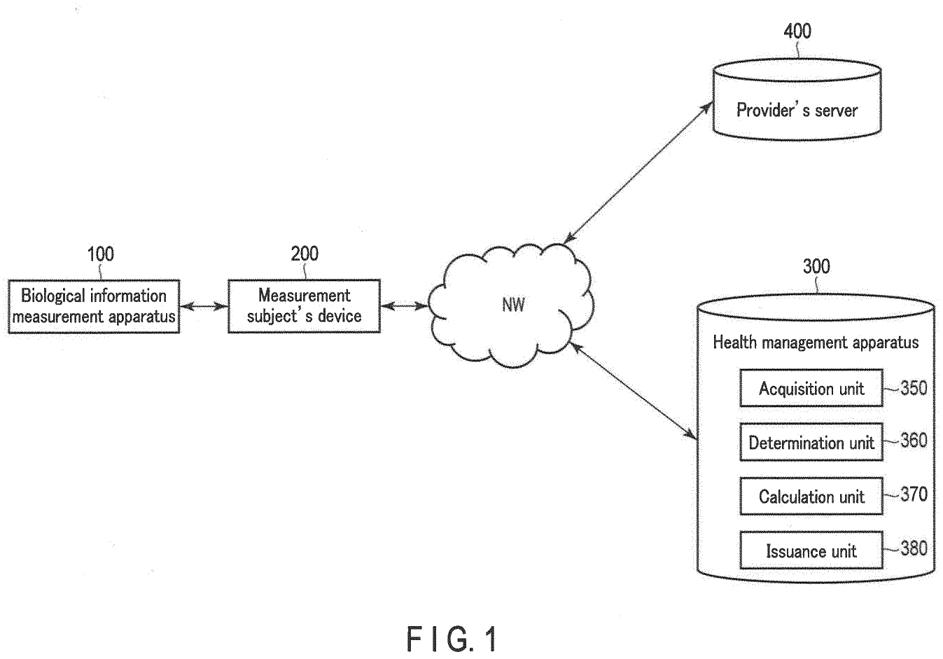

First, an example of an application of the present invention will be described with reference to FIG. 1.

As shown in FIG. 1, the health management system includes a biological information measurement apparatus 100, a measurement subject's device 200, a health management apparatus 300, and a provider's server 400. The biological information measurement apparatus 100 and the health management apparatus 300 are connected to each other in a communicatory manner via the measurement subject's device 200. The measurement subject's device 200 and the provider's server 400 are connected to the health management apparatus 300 in a communicatory manner via a network NW.

The biological information measurement apparatus 100 measures biological information of a measurement subject (who is a subject of measurement), and transmits the information along with data and time of the measurement to the measurement subject's device 200. The biological information includes, for example, information related to body composition (such as a body weight, a body mass index (BMI), a body fat rate, muscle mass, bone mass, a basal metabolic rate, etc.) and information related to life activities (such as a blood pressure value and a physical activity amount). The biological information measurement apparatus 100 includes, for example, a body composition measurement apparatus capable of measuring body composition, or a blood pressure measurement apparatus capable of measuring a blood pressure value. The biological information measurement apparatus 100 may be either an installation-type device installed in a house, etc. or a wearable device that can be continuously worn by the measurement subject.

The measurement subject's device 200 is an information communication device used by the measurement subject, and includes, for example, a portable information communication device, such as a smart phone, a mobile phone, a tablet PC, or a laptop PC. The measurement subject's device 200 receives an input of goal setting information set by the measurement subject in order to improve their health situation, and transmits the information to the health management apparatus 300, in accordance with an operation by the measurement subject. The goal setting information includes a goal value relating to biological information, for example. Upon receipt of biological information from the biological information measurement apparatus 100, the measurement subject's device 200 transfers the biological information to the health management apparatus 300. The measurement subject's device 200 receives reward points or a coupon(s) issued by the health management apparatus 300. The measurement subject's device 200 receives provision of services from the provider's server 400 based on the biological information.

The biological information measurement apparatus 100 may only have a function of measuring biological information, and does not necessarily have a function of transmitting the biological information to the measurement subject's device 200. In this case, the measurement subject's device 200 may receive an input of biological information measured by the biological information measurement apparatus 100 in accordance with an operation of the measurement subject, and may transmit the biological information to the health management apparatus 300.

The health management apparatus 300 is a server apparatus comprising a function configuration for accustoming the measurement subject to measuring their own biological information, and for promoting improvement in a health situation of the measurement subject. The health management apparatus 300 includes an acquisition unit 350, a determination unit 360 a calculation unit 370, and an issuance unit 380.

The acquisition unit 350 acquires biological information and goal setting information of the measurement subject. The goal setting information is set in the health management apparatus 300 as goal management information including a goal value that should be attained within a predetermined period of time. The determination unit 360 determines whether or not the biological information acquired by the acquisition unit 350 attains the goal value set with respect to the biological information within the predetermined period of time. The calculation unit 370 calculates an incentive value of the measurement subject based on a determination, result obtained by the determination unit 360. The issuance unit 380 issues at least either reward points or a coupon(s) to the measurement subject based on the incentive value calculated by the calculation unit 370.

The health management apparatus 300 integrates the biological information acquired from the measurement subject, and provides the information to the provider's server 400.

The provider's server 400 is a server apparatus used by a service provider via the use of the biological information acquired from the measurement subject, for example. The service provider includes an insurance company or a health insurance association, for example. The provider's server 400 uses the biological information integrated by the health management apparatus 300 to calculate insurance, and provides insurance services to the measurement subject.

As described above, the health management apparatus 300 determines whether or not biological information, acquired from the measurement subject attains a goal value set by the measurement subject. The health management apparatus 300 calculates an incentive value in accordance with whether or not the goal value is attained. It is thereby possible for the measurement subject to receive reward points or a coupon(s) in accordance with whether or not the goal value is attained. It is thus possible to motivate the measurement subject to attain the goal value so that they can obtain a higher incentive,

Now being motivated to attain the goal value, the measurement subject comes to continuously measure their biological information so as to check whether or not the goal value is attained. It is thus possible to accustom the measurement subject to measuring biological information.

The health management apparatus 300 can acquire the increased number of biological information items through accustoming the measurement subject to measuring their biological information. It is thus possible for the health management apparatus 300 to provide more biological information items to the provider's server 400. A service provider can provide services of higher quality through the use of the increased number of biological information items for their services. It is thus possible for the measurement subject to receive services of higher quality from a service provider.

### 2. Embodiment

An Embodiment of the health management apparatus according to the foregoing applied example will be described below. In the following, a health management system including an apparatus, as an example of a biological information measurement apparatus, capable of measuring a body weight and body composition will be described.

### 2.1 Examples of Hardware Configuration

An example of a hardware configuration of each apparatus in the health management system according to the embodiment will be described.

### 2.1.1 Example of Hardware Configuration of Biological Information Measurement Apparatus

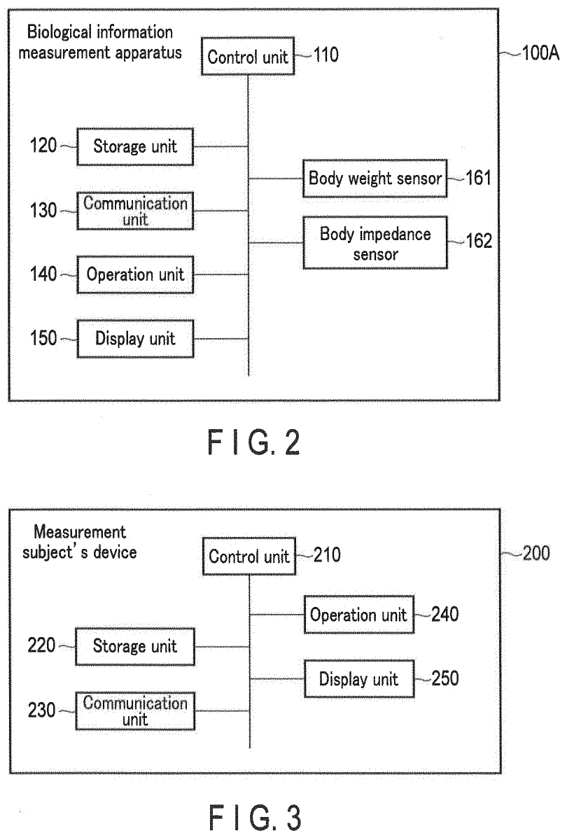

First, a hardware configuration example of the biological information measurement apparatus 100A according to the embodiment will be described. FIG. 2 is a block diagram showing a hardware configuration of a biological information measurement apparatus 100A according to the embodiment. As shown in FIG. 2, the biological information measurement apparatus 100A according to the embodiment includes a control unit 110, a storage unit 120, a communication unit 130, an operation unit 140, a display unit 150, a body weight sensor 161, and a body impedance sensor 162.

The control unit 110 includes a central processing unit (CPU), a random access memory (RAM), and a read only memory (ROM), etc., and controls each constituent element in accordance with information processing. The control unit 110 includes a clock (not shown), and has a clock function to display current date and time.

The control unit 110 generates body weight information and body composition information based on a measurement result obtained by the body weight sensor 161 and the body impedance sensor 162, and basic information relating to the measurement subject input through an operation of the operation unit 140 by the measurement subject themselves.

The basic information relating to the measurement subject includes, for example, age, sex, and a height, etc. of the measurement subject. The body weight information includes a body weight and a EMI of the measurement subject, for example. The body composition information includes a body fat rate, muscle mass, bone mass, and a basal metabolic rate of the measurement subject. In the following descriptions, the body weight information and the body composition information will be referred to as “body weight and composition information”. Since the body weight and composition information is one piece of biological information, the body weight and composition information may be simply referred to as “biological information” in the following description. The biological information is associated with the date and time of measurement indicated by the clock. The biological information may be further associated with basic information of the measurement subject, a device ID for uniquely identifying the biological information measurement apparatus 100A, and so on.

The storage unit 120 is an auxiliary storage apparatus, such as a hard disk drive (HDD) or a solid state drive (SSD). The storage unit 120 temporarily stores the biological information items until those information is transmitted to the measurement subject's device 200.

The communication unit 130 is a communication interface that administers communications with the measurement subject's device 200. The communication unit 130 transmits the biological information to the measurement subject's device 200, for example. In the embodiment, a short-distance communication, such as Bluetooth (registered trademark), is applicable to a communication between the communication unit 130 and the measurement subject's device 200, but not limited thereto. A communication via a network NW, such as a local area network (LAN), or a wired communication using a communication cable may be adopted for the communication by the communication unit 130.

The operation unit 140 includes a user interface, such as a touch panel and operation buttons, etc., for example. The operation unit 140 detects an operation by the measurement subject via the user interface, and outputs to the control unit 110 a signal indicating a type of the operation.

The display unit 150 includes, for example, a display screen (e.g., a liquid crystal display (LCD) or an electroluminescence (EL) display) and an indicator, etc. The display unit 150 displays information in accordance with a signal from the control unit 110, and notifies the information to the measurement subject.

The body weight sensor 161 is, for example, a distortion gauge, and measures a body weight of the measurement subject.

The body impedance sensor 162 measures a body impedance of the measurement subject, for example. The body impedance sensor 162 includes, for measuring body impedance, an electrode for applying a constant current to the body of the measurement subject and an electrode for measuring a voltage derived from the body when the current is applied.

### 2.1.2 Example of Hardware Configuration of Mobile Device

Next, the hardware configuration example of the measurement subject's device 200 will be described. FIG. 3 is a block diagram showing an example of a hardware configuration of the measurement subject's device 200 according to the embodiment. As shown in FIG. 3, the measurement subject's device 200 includes a control unit 210, a storage unit 220, a communication unit 230, an operation unit 240, and a display unit 250.

The control unit 210 and the storage unit 220 are respectively the same as the control unit 110 and the storage unit 120 of the biological information measurement: apparatus 100A. However, the control unit 210 of the measurement subject's device 200 generates goal setting information based on information input upon an operation of the operation unit 240 by the measurement subject. In addition, the storage unit 220 of the measurement subject's device 200 temporarily stores the goal setting information and the biological information received from the biological information measurement apparatus 100A, until these information items are transmitted to the health management apparatus 300.

The goal setting information is information set by the measurement subject for improving their health situation, and includes, for example, a goal value relating to biological information. The goal setting information may further include information indicating a time limit for attaining the goal value. Specifically, for example, the goal setting information includes information indicating “biological information (e.g., a body weight) attains a goal value (e.g., 60 kg) within a predetermined period of time (e.g., 30 days of the present time)”. The goal setting information is not limited to the above example, and a goal may be set in various ways. For example, biological information set in the goal setting information is not limited to a body weight, and any biological information can be set. For example, a goal value set in the goal setting information is not necessarily a specific numerical value, and a notion such as “bring the value to the standard value”, for example, may be set.

The communication unit 230 is a communication interface that administers communications between the biological information measurement apparatus 100A, the health management apparatus 300, and the provider's server 400. For example, the communication unit 230 receives biological information from the biological information measurement apparatus 100A. The communication unit 230 transmits biological information and the goal setting information to the health management apparatus 300, and receives reward points or coupon(s). The communication unit 230 may be provided with services from the provider's server 400. In the embodiment, for example a communication via a network NW is applicable to a communication between the health management apparatus 300 and the provider's server 400 administered by the communication unit 230; however, the communication is not limited thereto, and a short-distance wireless communication or a wired communication is also applicable.

The operation unit 240 and the display unit 250 are respectively the same as the operation unit 140 and the display unit 150 of the biological information measurement apparatus 100A.

### 2.1.3 Example of Hardware Configuration of Health Management Apparatus

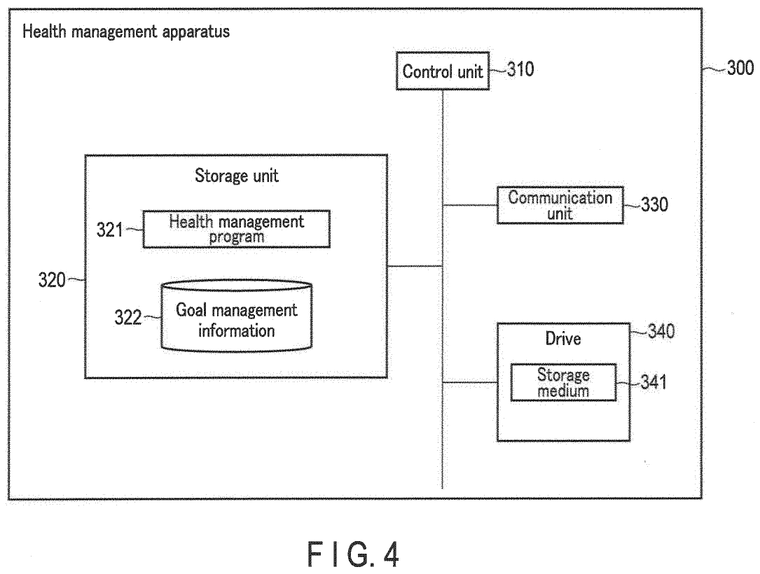

Next, the hardware configuration example of the health management apparatus 300 will be described. FIG. 4 is a block diagram showing an example of a hardware configuration of the health management apparatus 300 according to the embodiment. As shown in FIG. 4, the health management apparatus 300 includes a control unit 310, a storage unit 320, a communication unit 330, and a drive 340.

The control unit 310 and the storage unit 320 are respectively the same as the control unit 110 and the storage unit 120 of the biological information measurement apparatus 100A. The storage unit 320 of the health management apparatus 300 stores goal management information 322, etc. used in a health management program 321 executed in the control unit 310.

The communication unit 330 is a communication interface that administers communications between the measurement subject's device 200 and the provider's server 400. For example, the communication unit 330 receives the biological information and the goal setting information from the measurement subject's device 200. The communication unit 330 transmits, for example, a set of biological information items measured from the same measurement subject to the provider's server 400.

The communication unit 330 may receive a desired type of reward from the provider's server 400 in exchange for provision of the set of biological information items. Rewards may consist of a monetary reward, points, or a coupon(s), for example. The communication unit 330 may receive information regarding a health situation of the measurement subject from the provider's server 400, and may transmit a desired type of reward in exchange for the information. The information relating to a health situation of the measurement subject that may be received from the provider's server 400 includes, for example, a diagnosis result of a health checkup or a thorough medical checkup of the measurement subject, etc.

The drive 340 is a drive, such as a compact disk (CD) drive or a digital versatile disk (DVD) drive, etc., and is a device for reading programs stored in the storage medium 341. A type of the drive 340 may be selected as appropriate in accordance with a type of the storage medium 341. The health management program 321 and the goal management information 322 may be stored in the storage medium 341.

The storage medium 341 is a medium storing information of programs recorded thereon in an electronic, magnetic, optical, mechanical, or chemical manner so that the information of programs is readable for a computer, device or machine. The health management apparatus 300 may obtain the health management program 321 and the goal management information 322 from the storage medium 341.

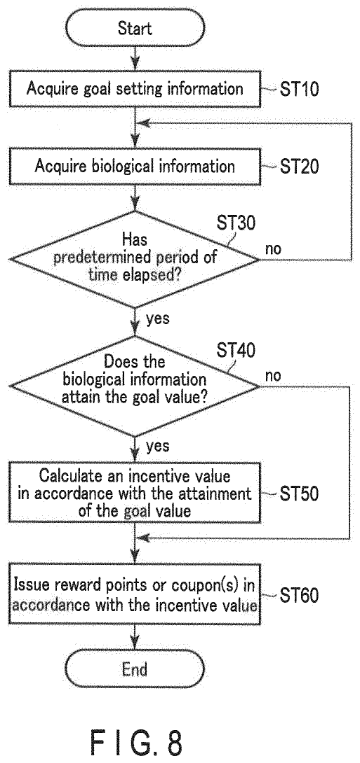

The health management program 321 is a program for managing a health situation of the measurement subject through determination of whether or not biological information attains a goal value within a period of time, and causing the health management apparatus 300 to perform the health management process (FIG. 8) in which reward points or a coupon(s) are issued in accordance with a result of the determination.

The goal management information 322 is information indicating a relationship between biological information obtained by the measurement subject and a set goal value. In the goal management information 322, a goal value, biological information acquired until a current point of time since the goal value is set, and a time limit for attaining the goal value are set based on the goal setting information obtained from the measurement subject's device 200. In the case where a notion of “bring the value to the standard value” is set as a goal value in the goal setting information, the control unit 310 sets a goal value which is preset as a standard value in the goal management information 322 based on the basic information relating to the measurement subject. The goal management information 322 is updated every time biological information is acquired between the point at which the goal value is set and the point at which a predetermined period of time for attaining the set goal value elapses.

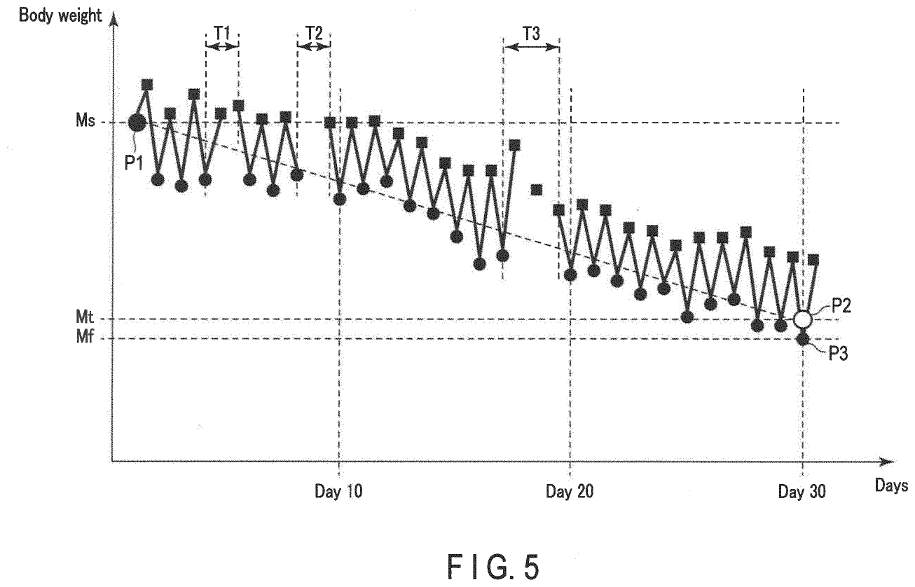

FIG. 5 is a diagram showing an example of the goal management information 322 according to the embodiment. The example of FIG. 5 shows a case where a body weight is acquired as biological information. In the example of FIG. 5, 30 days is set as a time limit for attaining the goal value, and the goal management information 322 at the time when the period of 30 days elapses is visually shown. Specifically, in FIG. 5, a number of days elapsed since the goal value is set is indicated on the horizontal axis, and a body weight as biological information is indicated on the vertical axis. In FIG. 5, the actually-acquired body weights are indicated as a black plot, and the body weight set as the goal value is indicated as a white plot. In FIG. 5, of the actually-acquired body weights, body weights acquired during a morning (for example, from the sunrise to noon) are indicated as a plot of round dots, and body weights acquired during a night (for example, from the sunset to midnight) are indicated as a plot of square dots.

As shown in FIG. 5, the goal management information 322 includes a point P1 indicating an initial value Ms at the time of setting the goal value, a point P2 indicating a goal value Mt determined based on the goal setting information, and a point P3 indicating a final value Mf at the time when the predetermined period of time elapses. The goal management information 322 further includes multiple points indicating body weights acquired within a period between the point P1 and the point P3. According to FIG. 5, through referring to the goal management information 322, the control unit 310 can determine that biological information is acquired twice, namely during a morning and at night, over the entire period of time of 30 days except for periods T1, T2, and T3. The control unit 310 can determine that the body weight of the measurement subject at the initial value Ms gradually becomes closer to the goal value Mt, and attains the goal value Mt on day 28.

### 2.1.4 Example of Hardware Configuration of Provider's Server

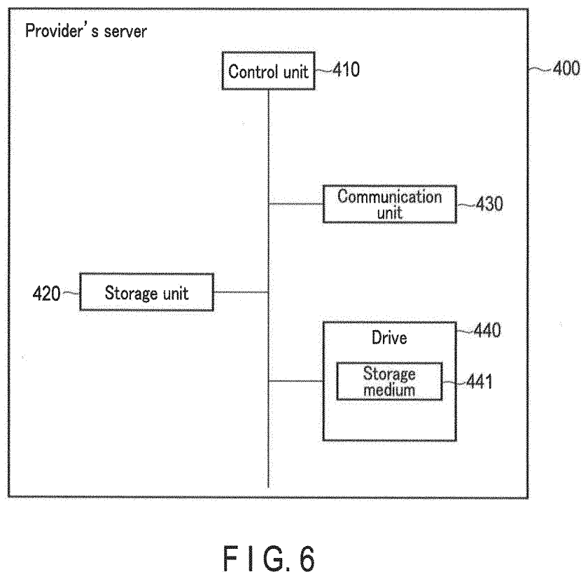

Next, the hardware configuration example of the provider's server 400 will be described. FIG. 6 is a block diagram showing an example of a hardware configuration of the provider's server 400 according to the embodiment. As shown in FIG. 6, the provider's server 400 includes a control unit 410, a storage unit 420, a communication unit 430, and a drive 440. The drive 440 is an apparatus for reading programs stored in the storage medium 441.

The control unit 410 and the storage unit 420 are respectively the same as the control unit 310 and the storage unit 320 of the health management apparatus 300. The storage unit 420 of the provider's server 400 stores services, etc. (not shown).

The communication unit 430 is a communication interface that administers communications between the measurement subject's device 200 and the health management apparatus 300. For example, the communication unit 430 receives a set of biological information items from the health management apparatus 300. The communication unit 430 provides a service generated using the set of biological information to the measurement subject's device 200.

The drive 440 and the storage medium 441 are respectively the same as the drive 340 and the storage medium 341 of the health management apparatus 300.

### 2.2 Example of Function Configuration

Next, an example of a function configuration of the health management system according to the embodiment will be described.

### 2.2.1 Example of Function Configuration of Health Management Apparatus

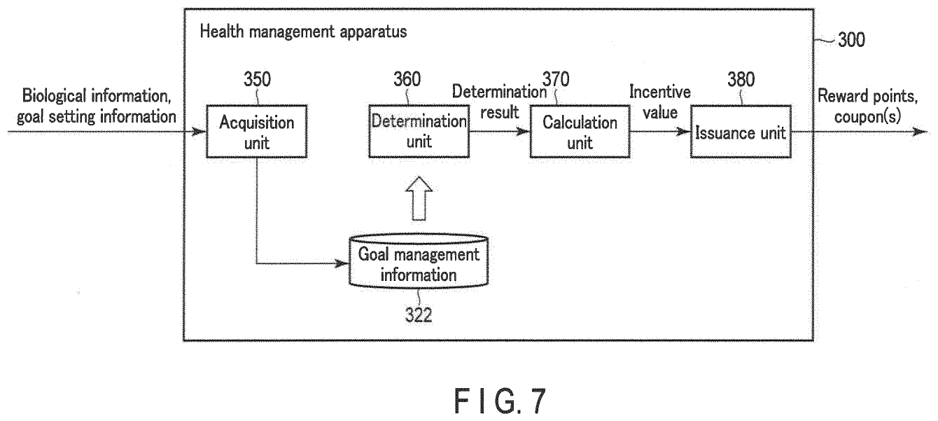

FIG. 7 is a block diagram schematically showing an example of a function configuration of the health management apparatus 300 of the health management system according to the embodiment.

The control unit 310 of the health management apparatus 300 loads the health management program 321 stored in the storage unit 320 in the RAM. Then, the control unit 310 interprets and executes, courtesy of the CPU, the health management program 321 loaded in the RAM to control each constituent element. Thus, as shown in FIG. 7, the health management apparatus 300 according to the embodiment functions as a computer that includes the acquisition unit 350, the determination unit 360, the calculation unit 370, and the issuance unit 380.

The acquisition unit 350 acquires the biological information measured by the biological information measurement apparatus 100A and the goal setting information set by the measurement subject's device 200, and transfers the information to the storage unit 320. The transferred biological information and goal setting information are integrated and stored as goal management information 322, for example.

The determination unit 360 reads the goal management information 322, and determines whether or not the acquired biological information attains a goal value within a predetermined period of time. The determination unit 360 generates a determination result, and transmits it to the calculation unit 370. When making the determination regarding the attainment, the determination unit 360 may compare only the biological information acquired at the last of the predetermined period of time with the goal value; however, the determination can be made in a different manner. For example, the determination unit 360 may compare an average of a predetermined number of biological information items acquired within the predetermined period of time (for example, items acquired in the last of the predetermined period of time) with the goal value.

Upon receipt of the determination result from the determination unit 360, the calculation unit 370 calculates an incentive value in accordance with the determination result. The calculation unit 370 transmits the calculated incentive value to the issuance unit 380.

Upon receipt of the incentive value from the calculation unit 370, the issuance unit 380 converts the incentive value into reward points or a coupon(s) correspondingly. The issuance unit 380 issues the reward points or coupon(s) obtained through the conversion of the incentive value to the measurement subject. The reward points or a coupon(s) are transmitted to, for example, the measurement subject's device 200.

### 2.3 Example of Operation

Next, an example of an operation of the health management system according to the embodiment will be described. The process procedure described hereinafter is merely an example, and the process can be changed as far as possible. Omission, replacement, or addition of a step in the process procedure described hereinafter can be made as appropriate, in accordance with an actual situation where the embodiment is realized.

### 2.3.1 Example of Operation of Health Management Apparatus

FIG. 8 is a flow chart schematically showing an example of a health management process in the health management apparatus 300 according to the embodiment.

(Step ST10)

In step ST10, the control unit 310 causes the communication unit 330 to function as the acquisition unit 350, and the communication unit 330 acquires the goal setting information from the measurement subject's device 200. The control unit 310 sets an initial value of the biological information of the acquired goal setting information, a goal value, and a time limit for attaining the goal value in the goal management information 322, and causes the storage unit 320 to store the values and the time limit. The goal value set by the measurement subject is thereby managed by the health management apparatus 300.

(Step ST20)

In step ST20, the control unit 310 causes the communication unit 330 to function as the acquisition unit 350, and the communication unit 330 acquires biological information measured within the predetermined period of time via the measurement subject's device 200, and causes the storage unit 320 to store the acquired biological information. The control unit 310 updates the goal management information 322 every time biological information is acquired.

(Step ST30)

In step ST30, the control unit 310 determines whether or not the predetermined period of time elapses based on the time limit set in the goal management information 322. If it is determined that the predetermined period of time has not yet elapsed (No in step ST30), the process proceeds to step ST20 and the control unit 310 acquires most recent biological information. If it is determined that the predetermined period of time has elapsed (Yes in step ST30), the control unit 310 finishes acquiring biological information, and the process proceeds to step ST40.

(Step ST40)

In step ST40, the control unit 310 functions as the determination unit 360, and determines whether or not the biological information attains the goal value within the predetermined period of time by referring to the goal management information 322. If it is determined that the biological information does not attain the goal value (No in step ST40), the control unit 310 does not calculate an incentive value for the measurement subject in exchange for attaining the goal value, and the process proceeds to step ST60. If it is determined that the biological information attains the goal value (Yes in step ST40), the process proceeds to step ST50 so that the control unit 310 calculates an incentive value.

(Step ST50)

In step ST50, the control unit 310 functions as the calculation unit 370 in accordance with the determination result in step ST40, and calculates an incentive value for the measurement subject in exchange for attaining the goal value.

(Step ST60)

In step ST60, the control unit 310 functions as the issuance unit 380, and, after converting the incentive value calculated in step ST50 into reward points or a coupon(s), issues the reward points or coupon(s) to the measurement subject's device 200. If an incentive step is not calculated in step ST50 because the goal value has not yet been attained, the control unit 310 need not issue reward points or a coupon(s).

The operation of the health management apparatus 300 is thus finished.

### 2.4 Advantageous Effects

According to the embodiment, technologies to build a routine of biological information measurement and promote improvement in a health situation are provided. This advantageous effect will be described below.

In the embodiment, in step ST40, after the predetermined period of time has elapsed since the goal value was set, it is determined whether or not the biological information acquired from the measurement subject attains the goal value. If the goal value is attained within the predetermined period of time, an incentive value is calculated in step ST50 in accordance with the attained goal value, and reward points or a coupon(s) are issued to the measurement subject in step ST60 in accordance with the calculated incentive value. Thus, the measurement subject can receive more points or coupons if the goal value is attained. As a consequence, the measurement subject comes to make efforts to attain the goal value, and continue measuring their biological information. Thus, the measurement subject is accustomed to conducting health management on their own. It is thus possible to accustom the measurement subject to measuring their biological information as well as improve their health situation.

The biological information is important information for quantifying risks of brain and cardiovascular diseases of the measurement subject, for example. For this reason, the biological information acquired by the health management apparatus 300 may have economic values for service providers specialized in services relating to such risks, for example health insurance. In the embodiment, the health management apparatus 300 can acquire the increased number of biological information items through accustoming the measurement subject to measuring their biological information. For this reason, the health management apparatus 300 can provide the increased number of biological information items to the provider's server 400. It is thereby possible for the service provider who manages the provider's server 400 to provide services of a higher quality to the measurement subject through using the biological information items provided from the health management apparatus 300.

### 3. Modifications

Although the embodiment of the present invention has been described in detail in the foregoing, the description is merely an example of the present invention in every respect. Various improvements and modifications can of course be made to the embodiment to the extent that no deviation is made from the scope of the present invention. The following modifications can be made for example. In the following, the same reference numerals are used for the same constituent elements of the foregoing embodiment, and redundant descriptions are omitted as appropriate. The following modifications can be combined as appropriate.

### 3.1 First Modification

In the foregoing embodiment, a case where the calculation unit 370 calculates an incentive value based on whether or not the goal value is attained within a predetermined period of time is described; however, he embodiment is not limited thereto.

Specifically, the calculation unit 370, for example, may calculate an incentive value further based on the number of biological information items acquired within a predetermined period of time. Specifically, the calculation unit 370, for example, may calculate an incentive value in proportion to the number of the biological information items, and may further add the incentive value in a gradual manner if the number of biological information items reaches a predetermined threshold value.

The number of biological information items that contributes to the calculation of an incentive value may be the number of biological information items determined to be valid among all the biological information items acquired within a predetermined period of time. In this case, the determination unit 360 further determines whether or not the acquired biological information items are valid. After determining whether or not the biological information items are valid, the determination unit 360 calculates the number of the valid biological information items, and transmits this number to the calculation unit 370.

Specifically, the determination unit 360 determines the validity of the most recent biological information item by comparing the time and date of measuring the most-recent item with the time and date of measuring the immediately-preceding valid biological information. More specifically, the determination unit 360 determines that the most-recent biological information is valid if the date and time of the item is apart from the date and time of the immediately-preceding valid biological information by a certain period of time (e.g., a half day), and determines that the most-recent biological information is not valid if those dates and times are not apart by the certain period of time. The determination unit 360 determines, for example, whether or not the date and time of the most-recent biological information is measured within the predetermined period of time, thereby determining validity of the most-recent biological information.

The determination unit 360 may add determination items as appropriate, in addition to the above-described example. For example, the determination unit 360 may determine validity based on whether or not the most-recent biological information is associated with a device ID of the biological information measurement apparatus 100A used by the measurement subject.

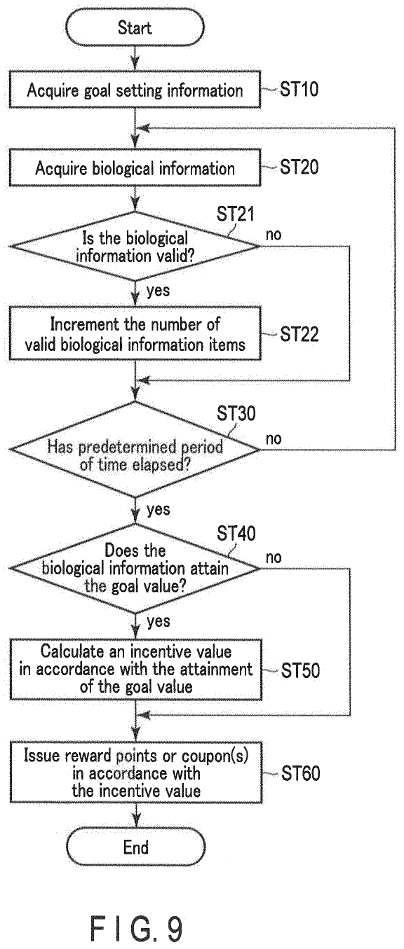

FIG. 9 is a flow chart schematically showing an example of an operation of the health management apparatus 300 according to the first modification of the embodiment. In FIG. 9, in addition to the operation described with reference to FIG. 8, steps ST21, ST22, and ST31 are added for calculating an incentive value further based on the number of valid biological information items.

Since the operations in step ST10 and step ST20 are the same as those shown in FIG. 8, descriptions are omitted.

(Step 5T21)

In step ST21, the control unit 310 functions as the determination unit 360, and determines whether or not a most-recently acquired biological information item is valid. If it is determined that the biological information item is not valid (No in step ST21), the process proceeds to step ST30. If it is determined that the biological information item is valid (Yes in step ST21), the process proceeds to step ST22.

(Step ST22)

In step ST22, the control unit 310 continuously functions as the determination unit 360, and, after incrementing the number of valid biological information items, causes the storage unit 320 to store the incremented number of valid biological information items.

Since the operation in step ST30 is the same as that shown in FIG. 8, descriptions of the step are omitted.

(Step ST31)

In step ST31, the control unit 310 functions as the calculation unit 370, and calculates an incentive value in accordance with the number of valid biological information items.

Since the operations in step ST40 and step ST50 are the same as those shown in FIG. 8, descriptions of those steps are omitted.

(Step ST60)

In step ST60, the control unit 310 functions as the issuance unit 380, and, after converting a sum of the incentive value calculated in steps ST31 and ST50 into reward points or a coupon(s), issues the reward points or coupon(s) to the measurement subject's device 200.

The operation of the health management apparatus 300 is thus finished.

It is thus possible for the health management apparatus 300 to give the measurement subject incentives in accordance with not only whether or not the goal value is attained but also the number of biological information items acquired during the process of attaining the goal value. For this reason, the measurement subject can be motivated to continuously provide biological information during the process of attaining the goal value.

Through the continuous provision of biological information during the process of attaining the goal value from the measurement subject, the health management apparatus 300 can acquire a set of biological information items during the process of attaining the goal value. It is thus possible for the health management apparatus 300 to provide a set of biological information items to the provider's server 400. The set of biological information items may have a value higher than biological information merely indicating whether or not the goal value is attained. For example, when the goal value cannot be attained, a service provider can know changes that have occurred in the biological information of the measurement subject based on the set of biological information items. For this reason, the service provider can enhance service quality based on the set of biological information items.

### 3.2 Second Modification

In the foregoing embodiment, a case where a final goal value within a predetermined period of time is set in the goal management information 322 is described; however, the present embodiment is not limited thereto. An intermediate goal value at any discretional point of time within a predetermined period of time may be set.

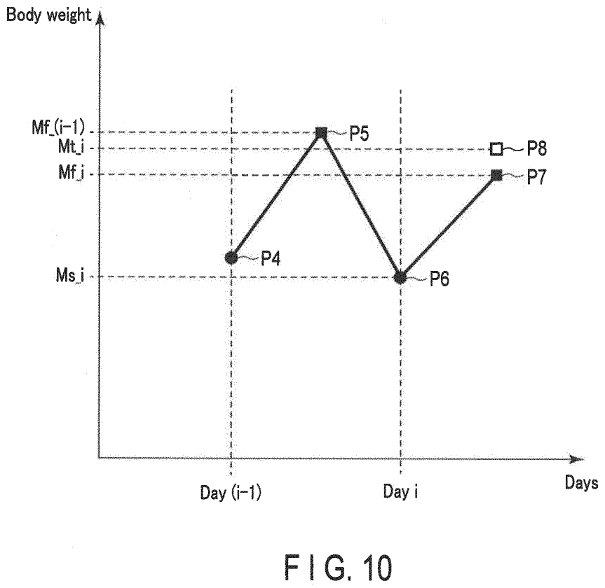

FIG. 10 is a diagram showing goal management information according to a second modification of the embodiment. FIG. 10 shows a part of the goal management information shown in FIG. 5, and shows biological information items acquired on day (i-1) and day i since the goal value is set and, an intermediate goal value set on day i (i is any natural number). Specifically, FIG. 10 shows points P4 and P5 respectively indicating body weights acquired during a morning and a night of a particular day (i-1), and points P6 and P7 respectively indicating body weights acquired during a morning and a night of a particular day i. FIG. 10 shows a point P8 indicating an intermediate goal value Mt_i of the body weight for the night of day i.

Generally, a body weight at night tends to be heavier than a body weight during a morning of the same day because of the weight of food consumed during the day. Furthermore, a body weight during a morning of the following day tends to be lighter than a body weight of a night before because of basal metabolic activities which take place during the night, Accordingly, to achieve weight reduction, it is desirable to restrict the an amount of increase (Mf_i−Ms_i) obtained by subtracting the body weight Mf_i during a night of day i from the body weight Ms_i during the morning of the day i to be lower than, for example, an amount of reduction (Mf_(i-1)−Ms_i) obtained by subtracting the body weight Ms_i during the morning of day i from the body weight Mf_(i-1) during the night of day (i-1). For this reason, the intermediate goal value Mt_i of day i is, for example, determined based on point P5 indicating the value Mf (i-1) and point P6 indicating Ms_i, and is compared with point P7 indicating the value Mf_i.

The determination unit 360 reads the goal management information 322, and determines whether or not the acquired biological information attains the intermediate goal value set correspondingly to a point of time in a predetermined period of time. The determination unit 360 generates a determination result, and transmits it to the calculation unit 370. In the example of FIG. 10, since the value Mf_i is smaller than the value Mt_i, it is determined that the intermediate goal value set on day i is attained.

Upon receipt of the determination result regarding the intermediate goal value from the determination unit 360, the calculation unit 370 calculates an incentive value in accordance with the determination result. The calculation unit 370 sums the incentive value calculated for the intermediate goal value, and the incentive value regarding the final goal value, and transmits the sum to the issuance unit 380.

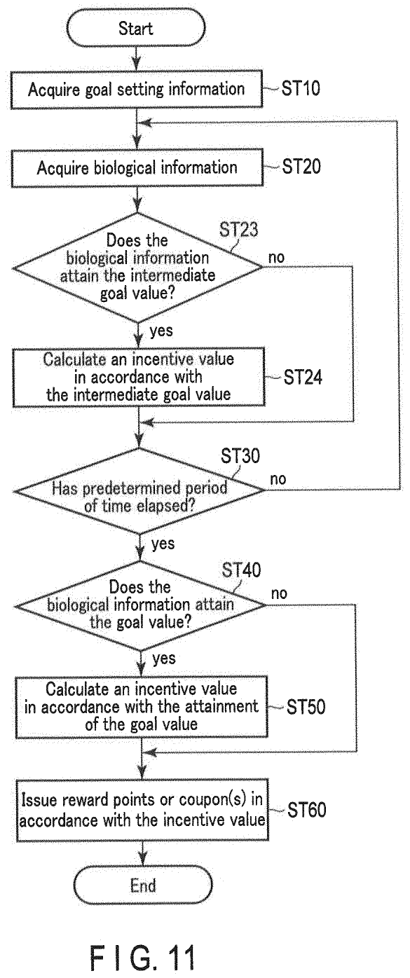

FIG. 11 is a flow chart schematically showing an example of an operation of the health management apparatus 300 according to the second modification of the embodiment. In FIG. 11, in addition to the operation described with reference to FIG. 8, steps ST23 and ST24 for calculating an incentive value further based on whether or not the intermediate goal value is attained are added.

Since the operations in step ST10 and step ST20 are the same as those shown in FIG. 8, descriptions of those steps are omitted.

(Step ST23)

In step ST23, the control unit 310 determines whether or not the biological information attains the intermediate goal value by referring to the goal management information. 322. If it is determined that the biological information does not attain the intermediate goal value (No in step ST23), the control unit 310 does not calculate an incentive value in exchange for attaining the intermediate goal value for the measurement subject, and the process proceeds to step ST30. If it is determined that the biological information attains the intermediate goal value (Yes in step ST23), the process proceeds to step ST24 so that the control unit 310 calculates an incentive value.

(Step ST24)

In step ST24, the control unit 310 functions as the calculation unit 370 in accordance with a determination result in step ST23, and calculates an incentive value in exchange for attaining the intermediate goal value for the measurement subject.

Since the operations in steps ST30, ST40, and ST50 are the same as those shown in FIG. 8, descriptions of those steps are omitted.

(Step ST60)

In step ST60, the control unit 310 functions as the issuance unit 380, and, after converting a sum of the incentive value calculated in steps ST24 and ST50 into reward points or a coupon(s), issues the reward points or coupon(s) to the measurement subject's device 200.

The operation of the health management apparatus 300 is thus finished.

It is thus possible for the health management apparatus 300 to set an intermediate goal value during the process of attaining the final goal value, and to calculate an incentive value in accordance with the attained intermediate goal value. For this reason, the measurement subject can be motivated not only to attain the final goal value but also to pay attention to the improvement of their biological information during the process. Accordingly, it is possible to further advance the health management capabilities of the measurement subject.

### 3.3 Third Modification

In the foregoing embodiment, an apparatus capable of measuring body composition information is adopted as the biological information measurement apparatus 100; however, the embodiment is not limited thereto. For example, an apparatus capable of measuring a blood pressure value, etc. can be adopted as the biological information measurement apparatus 100. If an apparatus capable of measuring a blood pressure value is adopted as the biological information measurement apparatus 100, the health management apparatus 300 may further acquire health information indicating a health situation of the measurement subject. The health management apparatus 300 may determine that biological information having a higher value is acquired if the acquired blood pressure value satisfies a predetermined condition based on the health information, and consequently calculate more incentive values.

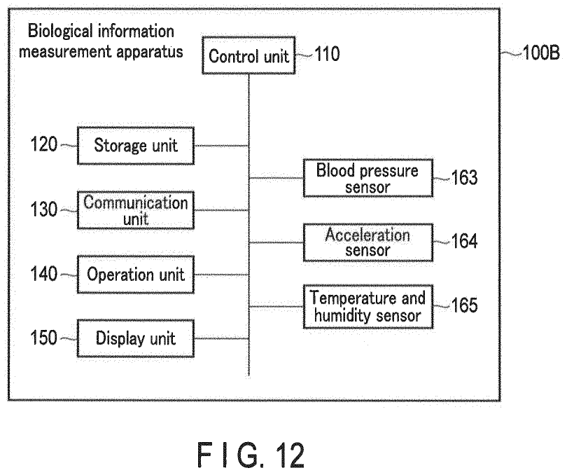

FIG. 12 is a block diagram showing a hardware configuration of a biological information measurement apparatus according to the third modification of the embodiment. The biological information measurement apparatus 100B shown in FIG. 12 is an apparatus capable of measuring a blood pressure value, etc. as biological information, for example a wearable device that can be attached/detached by the measurement subject. The biological information measurement apparatus 100B includes a control unit 110, a storage unit 120, a communication unit 130, an operation unit. 140, a display unit 150, a blood pressure sensor 163, an acceleration sensor 164, and a temperature and humidity sensor 165.

The control unit 110, the storage unit 120, the communication unit 130, the operation unit 140, and the display unit 150 are respectively the same as those of the biological information measurement apparatus 100A. The control unit 110 of the biological information measurement apparatus 100B, however, generates blood pressure information, activity information, and environment information based on measurement results obtained by the blood pressure sensor 163, the acceleration sensor 164, and the temperature and humidity sensor 165. The blood pressure information includes a blood pressure value of the measurement subject based on the measurement by the blood pressure sensor 163. The activity information includes a physical activity amount, the number of steps, and a sleeping condition of the measurement subject, based on the measurement by the acceleration sensor 164. The environment information includes temperature and humidity of the environment of the measurement subject based on the measurement by the temperature and humidity sensor 165. Each of the blood pressure information, the activity information, and the environment information is associated with the time and date of the measurement indicated by a clock. Each of the blood pressure information, the activity information, and the environment information may be further associated with a device ID for uniquely identifying the biological information measurement apparatus 100B.

The control unit 110 generates blood pressure information, etc. based on basic information of the measurement subject that is input through the operation of the operation unit 140 by the measurement subject. The basic information of the measurement subject may further include, for example, health information indicating a health situation of the measurement subject. The health situation of the measurement subject include, for example, a symptom relating to hypertension of the measurement subject. For example, the health information includes information indicating whether or not the measurement subject has sleep apnea syndrome (SAS), whether or not the measurement subject has morning hypertension, and whether or not the measurement subject has workplace hypertension, and the like.

The blood pressure sensor 163 measures a blood pressure value of the measurement subject. The blood pressure value includes typical indices of a highest blood pressure and a lowest blood pressure, for example. The blood pressure sensor 163 may be, for example, a continuous-measurement type capable of measuring blood pressure of the measurement subject per heartbeat (namely, in a continuous manner), or a non-continuous-measurement type capable of measuring blood pressure for a predetermined timing on the spot (namely, in a non-continuous manner). For the blood pressure sensor 163 of the continuous-measurement type, a method of continuously measuring blood pressure of the measurement subject based on a pulse transmit time (PTT) or a method of continuously measuring blood pressure based on a pressure pulse wave (the tonometry method) are applicable. The procedure of continuously measuring blood pressure is not limited to the above procedure; a procedure of detecting pulse wave using a light-emitting device can be adopted as appropriate. As the blood pressure sensor 163 of the non-continuous measuring type, for example, a procedure of detecting a pulse wave through pressing blood vessels using a cuff as a pressure sensor (the oscillometric method) can be adopted.

The acceleration sensor 164 detects an acceleration rate of the measurement subject which is caused at a place where the biological information measurement apparatus 100B is attached as a set of triaxial components. The acceleration sensor 164 may further include a gyro sensor, and may further detect angular velocity as a set of triaxial components in addition to the acceleration rate.

The temperature and humidity sensor 165 measures temperature and humidity of the environment of the measurement subject.

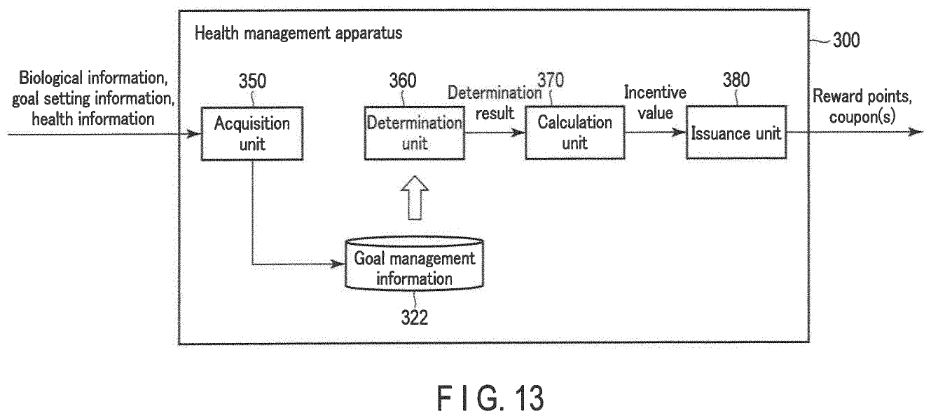

FIG. 13 is a block diagram showing a function configuration of a health management apparatus according to the third modification of the embodiment.

As shown in FIG. 13, the acquisition unit 350 acquires biological information, health information, and goal setting information via the measurement subject's device 200, and transfers the information to the storage unit 320. The biological information, health information, and goal setting information are integrated and stored as goal management information 322, for example.

The determination unit 360 determines whether or not the biological information satisfies a predetermined condition based on the health information. The determination unit 360 generates a determination result, and transmits it to the calculation unit 370.

Generally, a measurement subject who has a symptom of hypertension wants to know their blood pressure value when the value is high. For this reason, the blood pressure value acquired at a time of day when the blood pressure value is high is more valuable. On the other hand, a time of day at which a blood pressure value becomes high may vary depending on a health situation of a measurement subject. For this reason, whether or not the acquired blood pressure value is more valuable may be individually determined in accordance with a health situation of the measurement subject.

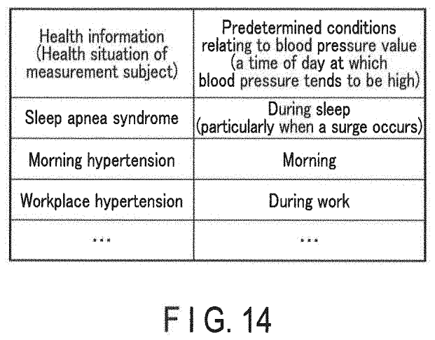

FIG. 14 is a table showing a relationship between a health situation of the measurement subject and a time of day at which blood pressure tends to be high according to the third modification of the present embodiment.

As shown in FIG. 14, if the measurement subject has sleep apnea syndrome for example, the blood pressure value is likely to be high during sleep, particularly when a surge occurs. If the measurement subject has morning hypertension, their blood pressure value is likely to become high during a morning. If the measurement subject has workplace hypertension, their blood pressure value is likely to become high during work time.

For this reason, the determination unit 360 determines whether or not the time at which the blood pressure value is acquired is a time of day at which blood pressure becomes high, in accordance with a health situation of the measurement subject. If the blood pressure value is acquired during the time of day at which blood pressure tends to high, it is determined that the value satisfies the predetermined condition; if the value is not acquired during the time of day at which blood pressure tends to be high, it is determined that the value does not satisfy the predetermined condition.

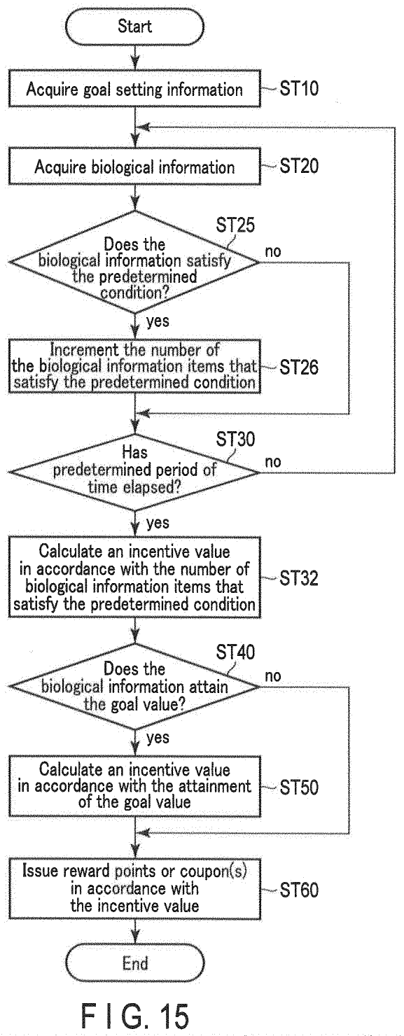

FIG. 15 is a flow chart schematically showing an example of a procedure of the health management process in the health management apparatus according to the third modification of the embodiment.

In FIG. 15, in addition to the operation described with reference to FIG. 8, steps ST25, ST26, and ST32 are added for calculating an incentive value further based on whether or not biological information satisfies a predetermined condition based on health information.

Since the operations in step ST10 and step ST20 are the same as those shown in FIG. 8, descriptions of those steps are omitted.

(Step ST25)

In step ST25, the control unit 310 determines whether or not the biological information satisfies a predetermined condition based on the health information. If it is determined that the biological information does not satisfy the predetermined condition (No in step ST25), the process proceeds to step ST30. If it is determined that the biological information satisfies the predetermined condition (Yes in step ST25), the process proceeds to step ST26.

(Step ST26)

In step ST26, the control unit 310 continuously functions as the determination unit 360, and, after incrementing the number of biological information items that satisfy the predetermined condition, causes the storage unit 320 to store the incremented number of valid biological information items.

Since the operation in step ST30 is the same as that shown in FIG. 8, descriptions of the step are omitted.

(Step ST32)

In step ST32, the control unit 310 functions as the calculation unit 370, and calculates an incentive value in accordance with the number of biological information items that satisfy the predetermined condition.

Since the operations in step ST40 and step ST50 are the same as those shown in FIG. 8, descriptions of those steps are omitted.

(Step ST60)

In step ST60, the control unit 310 functions as the issuance unit 380, and, after converting a sum of the incentive value calculated in steps ST32 and ST50 into reward points or a coupon(s), issues the reward points or coupon(s) to the measurement subject's device 200.

The operation of the health management apparatus 300 is thus finished.

Thus, the health management apparatus 300 can give an incentive to the measurement subject in accordance with the number of biological information items acquired during a particularly important time of day, among all the biological information items acquired from the measurement subject who finds himself or herself in a particular health situation. For this reason, the measurement subject can be motivated to provide biological information measured during a particularly important time of day in accordance with their health situation. Specifically, if the measurement subject has symptoms of hypertension, the health management apparatus 300 can motivate the measurement subject to measure a blood pressure value during a time of day at which the value becomes particularly high.

### 3.4 Fourth Modification

In the third modification of the foregoing embodiment, a blood pressure value is set as a goal value; however, another index that can be calculated from a blood pressure value can be set as a goal value. For example, if the measurement subject has sleep apnea syndrome, an upper limit of the number of times when a surge occurs during sleep may be set as a goal value.

In this case, it is preferable to continuously measure a blood pressure value during sleep with the biological information measurement apparatus 100B so as to determine whether or not a surge occurs. Accordingly, the continuous-measurement type is more preferable than the non-continuous measurement type for the biological information measurement apparatus 100B.

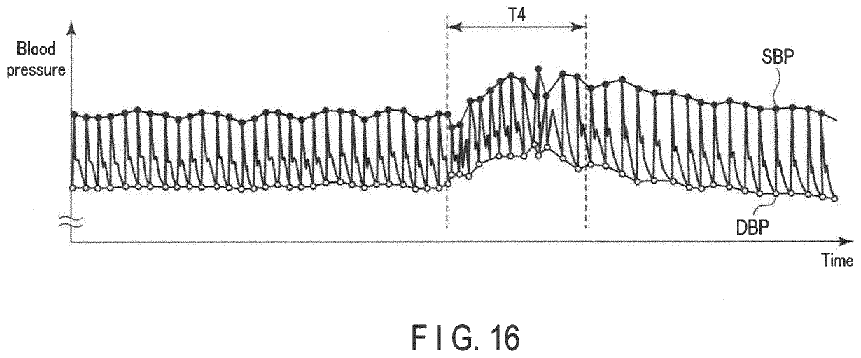

FIG. 16 is a diagram showing biological information according to a fourth modification of the embodiment. FIG. 16 shows a state of a blood pressure value continuously measured during sleep. Specifically, FIG. 16 shows points SBP and DBP as the highest blood pressure and lowest blood pressure per heartbeat. FIG. 16 shows a situation where a surge occurs in the period T4.

As shown in FIG. 16, in a normal state, the highest and lowest blood pressure values are stable for every heartbeat during sleep. However, once a surge occurs, both the highest and lowest blood pressures rise rapidly.

The acquisition unit 350 acquires biological information continuously measured over a night, as shown in FIG. 16, for a few days.

The determination unit 360 determines whether or not a surge occurs based on the acquired biological information. If it is determined that a surge occurs, the determination unit 360 increments the number of instances of surge occurrence, and then causes the storage unit 320 to store the incremented number of times of surge occurrence. The determination unit 360 may further determine whether or not the number of times of surge occurrence within the predetermined period of time is less than the upper limit set as the goal value.

If it is determined that the number of times of surge occurrence attains the goal value, the calculation unit 370 may calculate an incentive value in accordance with the determination result.

With the apparatus configured as described above, even if an evaluation item other than the acquired biological information is set as a goal value, an incentive value can be calculated as appropriate.

### 3.5 Others

In the foregoing embodiment, each of the functions of the health management apparatus 300 is included in one server apparatus; however, the embodiment is not limited to this example, and the functions may be separately included in multiple server apparatuses. For example, among the functions in the health management apparatus 300, the issuance unit 380 may be included in a server apparatus (for example, the provider's server 400) differing from the server apparatus that includes the acquisition unit 350, the determination unit 360, and the calculation unit 370.

In the foregoing embodiment, the health management apparatus 300 acquires biological information of the measurement subject via the biological information measurement apparatus 100; however, the embodiment is not limited to this example. For example, the health management apparatus 300 may acquire biological information of the measurement subject acquired by a service provider on their own from the provider's server 400. Even in this case, the operation of the health management apparatus 300 is no different to the acquisition of biological information from the measurement subject, although the acquisition is achieved through the business-to-business (“B2B”) exchange of biological information via the provider's server 400. For this reason, if the acquired biological information attains the goal value, the health management apparatus 300 may calculate an incentive value in accordance with the attainment of the goal value, and issue reward points or a coupon(s) to the measurement subject in accordance with the incentive value.

Furthermore, an example in which a blood pressure value or a body weight is used as biological information is described in the foregoing embodiment; however, the embodiment is not limited to this example. Other than this example, the present invention is applicable to examples where the biological information is pulse, body temperature, blood sugar value, or cardiogram, and the like.

The present invention is not limited to the above-described embodiment, and can be modified in practice, without departing from the gist of the invention. The respective embodiments may be appropriately combined and practiced. In this case, a combined effect is obtained. The above-described embodiment incorporates various kinds of inventions, and various kinds of inventions can be extracted by combinations selected from the plurality of disclosed constituent elements. For example, even if some constituent elements are deleted from all the constituent elements disclosed in the embodiment, an arrangement from which some constituent elements are deleted can be extracted as an invention as long as the problem can be solved and the effect can be obtained.

What is claimed is:

1. A health management apparatus comprising
an acquisition unit configured to acquire biological information of a measurement subject;
a determination unit configured to determine whether or not the acquired biological information attains a goal value set with respect to the biological information within a predetermined period of time; and
a calculation unit configured to calculate an incentive value of the measurement subject based on a determination result obtained by the determination unit, and a number of the biological information items, wherein
the acquisition unit further acquires health information indicating a health situation of the measurement subject,
the determination unit
sets a condition which includes a time of day at which blood pressure tends to be high in the biological information in accordance with the acquired health information, and
further determines whether or not the acquired biological information satisfies the set condition which includes a time of day at which blood pressure tends to be high, and
the calculation unit calculates the incentive value of the measurement subject further based on a number of biological information items that have been determined to satisfy the condition by the determination unit.

2. The health management apparatus according to claim 1, wherein
the determination unit further determines whether or not the acquired biological information is valid, and
the calculation unit calculates the incentive value of the measurement subject further based on a number of biological information items that have been determined to be valid by the determination unit.

3. The health management apparatus according to claim 1, wherein
the goal value includes at least one intermediate goal value set correspondingly to a point of time in the predetermined period of time, and
the determination unit further determines whether or not the acquired biological information attains the intermediate goal value at the point of time.

4. The health management apparatus according to claim 1, wherein
the biological information includes at least one of a blood pressure value and a body weight.

5. The health management apparatus according to claim 1, further comprising
an issuance unit configured to issue at least one of reward points and a coupon to the measurement subject based on the calculated incentive value.

6. A health management method performed by an apparatus for managing a health situation of a measurement subject, the method comprising:
a step of acquiring biological information of the measurement subject;
a step of determining whether or not the acquired biological information attains the goal value set with respect to the biological information within a predetermined period of time; and
a step of calculating an incentive value of the measurement subject based on a result of the determination and a number of the acquired biological information items, wherein
in the step of acquiring, health information indicating a health situation of the measurement subject is further acquired,
in the step of determining,
a condition which includes a time of day at which blood pressure tends to be high is set in the biological information in accordance with the acquired health information,
whether or not the acquired biological information satisfies the set condition which includes a time of day at which blood pressure tends to be high is further determined, and
in the step of calculating, the incentive value of the measurement subject is calculated further based on a number of biological information items that have been determined to satisfy the condition.

7. A non-transitory computer readable medium storing a computer program which is executed by a computer to provide steps of:
acquiring biological information of the measurement subject;
determining whether or not the acquired biological information attains the goal value set with respect to the biological information within a predetermined period of time; and
calculating an incentive value of the measurement subject based on a result of the determination and a number of the acquired biological information items, wherein
in the step of acquiring, health information indicating a health situation of the measurement subject is further acquired,
in the step of determining,
a condition which includes a time of day at which blood pressure tends to be high is set in the biological information in accordance with the acquired health information,
whether or not the acquired biological information satisfies the set condition which includes a time of day at which blood pressure tends to be high is further determined, and
in the step of calculating, the incentive value of the measurement subject is calculated further based on a number of biological information items that have been determined to satisfy the condition.
